(19) United States
(12) Patent Application Publication
(10) Pub. No.: US 2019/0333407 A1
(43) Pub. Date: Oct. 31, 2019

(54) Tactile Input for Improving Physical Movement

(71) Applicant: Erin McGuire, Burbank, CA (US)

(72) Inventor: Erin McGuire, Burbank, CA (US)

(21) Appl. No.: 16/403,578

(22) Filed: May 5, 2019

Related U.S. Application Data
(63) Continuation-in-part of application No. 14/994,054, filed on Jan. 12, 2016, now abandoned.

(51) Int. Cl. G09B 19/00; A61B 5/0488

(52) U.S. Cl. CPC G09B 19/003; A61B 2562/0247; A61B 5/0488

(57) ABSTRACT

A system for providing tactile input for prompting a person to coordinate appropriate muscle contractions during some form of physical movement such as exercising. The system may comprise a belt bearing inwardly facing projections adapted to make contact with a user's skin. The user may then be prompted by sensory tactile feedback or by audible, visible, or vibratory outputs to facilitate proper contractions, timing, and sequencing of activities, as may be appropriate given the type of physical activity. The projections are blunt, being hemispherical or hemispheroid, for example. The belt may include the projections, or alternatively, the projections may be attachable to a pre-existing belt. The projections may include transducers for providing signals to generate the audible, visible, or vibratory outputs. Outputs may be reproduced on a mobile communications device.

### CROSS-REFERENCE TO RELATED APPLICATIONS

This application is a Continuation Application of U.S. Non-Provisional application Ser. No. 14/994,054, entitled, “TACTILE INPUT FOR IMPROVING PHYSICAL MOVEMENT,” filed Jan. 12, 2016, the disclosure of which is incorporated herein by this reference.

### FIELD OF THE INVENTION

The present invention relates to the human body for the purpose of providing a means to improve muscle recruitment, movement, trunk stabilization function, respiratory function, exercising and sport performance, and the like. More specifically, the disclosure describes an apparatus having inwardly facing projections intended to provide sensory input to increase kinesthetic awareness which allows the user to improve the quality of muscle activity and contraction, including respiratory function, during physical movement and activity.

### DESCRIPTION OF THE PRIOR ART

Exercising devices are well known in the art and are designed for a wide variety of exercise modalities. More specifically, there are devices for exercising specific parts of the anatomy, there are devices for exercising the entire body, and there are biofeedback devices for measuring or monitoring movement and muscle action or force. However, at the present time, there is no such device that combines the foregoing functionalities while utilizing the stimulation of various sensory receptors (afferent inputs) as the primary means to stimulate and influence the Central Nervous System (CNS)) in order to effect a change.

U.S. Pat. No. 5,192,254 of Young teaches a sensor device to detect and signal the exercise or movement of a subject's craniofacial and cervical muscles that includes a trigger attached to the subject, sensitive to movement in at least one direction and communicating with a signal emitter in order to emit an appropriate signal upon the sensing of movement that indicates the performance of the exercise.

U.S. Pat. No. 5,474,083 of Church et al teaches an invention that is directed to a microprocessor based system utilizing electromyographic sensors to monitor muscle force for lift training and exercise training.

U.S. Pat. No. 6,059,576 of Brann teaches an electronic device, system and method to monitor and train an individual on proper motion during physical movement. The system employs an electronic device which tracks and monitors an individual's motion through the use of an accelerometer capable of measuring parameters associated with the individual's movement.

U.S. Pat. Pub. No. 2012/0094814 of Atkins et al teaches a method and apparatus for providing motional training, such as treatment of disequilibrium and movement and balance disorders, using cognitive spatial activity and by providing a subject with vibrotactile feedback in response to an attempt to perform the spatial activity.

U.S. Pat. Pub. No. 2014/0174174 of Uehara et al teaches a system and method for development of core muscles' support, comprising a means for identifying a user qualifying movement, a means for detecting a core muscle contraction in the identified qualifying movement, a means for discriminating between a core muscle contraction and no core muscle contraction in the identified qualifying movement; and a means to provide feedback to the user. Uehara is only concerned with sensing whether contraction has occurred and providing feedback.

Uehara tells the user if they have contracted their core or not, and the timing of when the contraction occurred relative to the movement; i.e. did it happen prior to the movement, which is how the core is supposed to work. In contrast the claimed device addresses the fact that there is a proper way the core muscles should contract which depends on proper timing and coordination of the core muscles and results in the expansion of the abdominal wall all the way around the trunk. When a poor pattern of core activation is present, it is difficult to change this coordination of core muscle activity because of engrained patterns in our brain and that sometimes, even with feedback provided by a device like this, the person will not be able to improve those engrained central nervous system patterns because they cannot feel how to change the muscle activity/coordination. No matter what cueing or information is given to them, if they cannot feel how to do it, their brain will continue to choose the pattern that it knows, even if it is faulty, because that is how it is wired. In order to re-educate the movement pattern and nervous system, they need some sort of sensory input to help them feel how to contract the muscles properly. This input is provided prior to the movement. In the case of the claimed device, it is done through pressure points that stimulate the receptors on the skin which helps increase the representation of that area in the brain to help the user better understand and feel how and where to contract. This novel device provides that benefit. As such Uehara does not disclose, teach, or suggest the idea of providing tactile input prior to contraction. The feedback provided by the Uehara apparatus, does not discern whether the contraction that occurred was of good or poor quality; only that a contraction occurred. The apparatus as claimed, relates to the proper coordination of muscle contraction. The device itself provides for proper muscle coordination through tactile input to stimulate the nervous system. The sensing of the muscle contraction is downstream from this novel feature.

Thus, while the foregoing body of prior art indicates it to be well known to use sensors, electrodes, electromyographic (EMG) sensors to monitor muscle activity and provide data to the user regarding said muscle activity after it has occurred, the prior art does not disclose a simple, highly portable, inexpensive, hands-free, device that provides sensory tactile input to stimulate the mechanoreceptors of the user prior to contraction, exercise, or activity in order to improve muscle activation and thereby improving stabilization, support, and movement while the activity or exercise is being executed. Nor does the prior art described above teach or suggest a hands-free device that stimulates the mechanoreceptors of the body to utilize neuromuscular and sensorimotor principles to facilitate and improve movement and the timing and coordination of said movement. Notably, the aforementioned body of prior art fails to provide any stimulus to the mechanoreceptors, or other prior to muscle contraction to assist a user, or patient, in the proper re-education of the neuromuscular movement patterns.

Further distinguishing characteristics of the present invention over the foregoing prior art will be made apparent from the following description thereof.

### BACKGROUND OF THE INVENTION

All purposeful movement requires proper movement and stability patterns. This applies to everyday activities such as walking, bending, reaching, breathing, etc., extending to physically demanding work environments, exercising, high level sports activities, and the like. These patterns are driven by our CNS and when optimal promote good quality coordination, and timing of muscle activity throughout our neuromuscular system. It is this optimal activity that aligns our joints, bones and muscles such that our bodies are supported and are best positioned to create integrated functional movement, power, and strength. Adoption of faulty patterns, or impaired coordination and quality of muscle activity, often occur for many reasons and is a prevalent issue in our society. Faulty ingrained movement patterns become automatic and unconscious, and therefore, occur unbeknownst to the individual as they move. When a faulty pattern is present and a movement is performed, i.e., reaching into a cabinet, a compensatory muscle coordination will occur resulting in the inadequate use of some muscles while other muscles are over used. The foregoing results in inefficiency of movement and imbalance around the joints and throughout the body, thereby placing excessive load on musculoskeletal structures, which can lead to pain, injury, disability and impaired performance.

Exercises have been developed to target specific muscles and/or muscle groups with the intention of providing rehabilitation, living a healthier lifestyle, and improving strength. One limitation to exercising is that the targeted muscles may or may not be activated as intended by any given exercise. As a result, the true benefit of the exercises is not realized. Performing a specific exercise meant to target a specific muscle or coordinated muscle activity will not necessarily result in the correct muscle activity or movement when compensatory muscle activity is present and CNS motor patterns have been altered. For instance, there are many exercises directed to strengthening the abdominal muscles of the trunk region. This region is an often targeted area of exercise and wellness because our ability to produce proper stabilization and support of our trunk and spine is a key factor in integrating movement throughout our body and movement system, allowing for optimal movement in our arms and legs, and in our overall physical health. Notably, proper trunk stabilization is achieved by the use of properly coordinated and timed contractions of the diaphragm, pelvic floor, deep segmental spinal muscles, deep neck flexors, deep spinal extensors, and the muscles of the abdominal wall. When the aforesaid muscles are correctly contracted the necessary increase in intra-abdominal pressure occurs, which stabilizes the spine and allows for healthy ideal movement.

While performing the many exercises directed to strengthening the abdominal muscles of the trunk region, there is often a lack of focus or awareness on the quality, timing and coordination of muscle activity and whether optimal function is occurring is unrealized. For example, it is common in the trunk or “core” muscles to see a compensatory pattern of excessive activity of the upper portion of the rectus abdominus and over activity of the lumbar and lower thoracic paraspinal muscles when a “core” exercise or movement that requires trunk or “core” stabilization is being performed. The result is poor stabilization function and an increase in compressive load on the spinal structures, among other things. In addition, strength and power production of the entire body will be negatively effected. In many cases such as this example, when altered CNS patterns exist, simply telling the individual to improve their core muscle activity by not over using their back muscles and increasing the use of their diaphragm muscle prior to contracting their abdominals will not be successful. When they attempt to change the activity, their brain uses the pattern it knows, which is the faulty one. At a more localized level looking at the quality of each specific muscle activity, the number of muscle fibers firing and the type of contraction occurring are important. In order to provide good quality stabilization or support function, whether we are looking at the trunk or other areas of the body, such as the scapula or hip, it is necessary for the contraction of the muscles responsible for that stabilization to be eccentric. When a good quality eccentric contraction occurs, it is typical to see an expansion or filling out of that muscle, vs a concentric contraction where you see the muscle become more narrow. In the instance of trunk stabilization, when proper coordination and timing occur, intra-abdominal pressure is increased so that if the proper eccentric activity of the abdominal wall follows, there will be an expansion of the entire area. This is important because EMG sensors and technology can only pick up muscle activity, it cannot differentiate between the type of contraction.

A pattern adopted by a person while they perform daily activities, play sports, lift, exercise, etc., is ingrained in the nervous system and is automatic. If improper, a person will need to relearn the pattern to correctly contract and coordinate the muscles involved, such as the trunk muscles. This re-education of movement patterns often requires specific input and facilitation to be provided so that the person will be able to retrain the faulty patterns that have become ingrained by being able to feel the correct movement or muscle contraction. Facilitation is any technique or input that makes movement easier or possible. After repeated conscious effort over time to re-learn the movement, the new pattern begins to become ingrained in the nervous system. Depending on the individual and the degree to which the pattern is ingrained, this can be a simple and short process or a lifelong effort.

In order to retrain a patient's muscle contractions, many physical therapists utilize methods to stimulate various receptors (afferent inputs) in order trigger the CNS, which is the part of the nervous system consisting of the brain and spinal cord. The CNS is the driver and controller of all human movement. This stimulation allows the physical therapist, or other professionals and practitioners involved with developing and training movement, i.e. the core, to fully utilize the neuroplasticity of the CNS and re-educate movement patterns. While this method is used for rehabilitation post injuries and surgeries, it is also extremely effective in helping a person improve the quality of their movement and trunk stabilization (which is crucial to proper and pain-free movement) during all human movement.

One type of sensory receptor, the mechanoreceptor, are nerve endings that are found in skin and muscle tissue. These receptors respond to mechanical stimuli such as touch, pressure, sound, vibration, and muscular contraction. It is important to note that there are different types of mechanoreceptors, i.e., Pacinian and Ruffini corpuscles, Merkel complex, etc. and that each of them respond to distinct types of information. While one responds to vibration, another will respond to deep pressure. There are also other key differences in the types of mechanical stimuli, in that vibration and simple touch are superficial stimuli and only involve the superficial layer of the skin. Whereas deep pressure involves a greater force and displaces underlying tissue in addition to the superficial layer of the skin. Some of these receptors are also slow or rapidly adapting. The receptors responsible for light touch and vibration are rapidly adapting which means the receptors do not continue firing to a constant stimulus and sensation will be felt for a short duration. But the receptors stimulated with deep pressure are slow adapting as the receptors continue sending impulses to the brain for a relatively long time allowing the sensation to be felt for a longer duration. When stimulated, by pressure, etc. these sensory receptors initiate nerve impulses in the afferent, or sensory neurons, which send the information to the CNS, or brain, which improves sensory awareness of that area and facilitates the corresponding muscle activity. The improved perception of the area occurs because the impulse travels to the portion of the brain that is attributed to the area of the body where the pressure or stimulus was applied. This leads to an increased ability to contract the muscles in said area resulting in improved motor control, muscle activation patterns and quality of muscle activation.

One way health care professionals stimulate the mechanoreceptors for the purpose of re-education, is to use fingers, either of the patient or of the health care professional tending to said patient. It is to be appreciated that “person,” “patient,” and “user” are interchangeable throughout the present disclosure. The fingers pressing upon a particular muscle provide tactile input, creating pressure and stimulating the mechanoreceptors. This increases the ability of the patient who is improperly activating the intended muscle/muscle group, for example trunk contractions, to have a better connection and sense of the area where an improved muscle activity is needed and thereby facilitates proper contraction and trunk stabilization.

In physical therapy, it is well known that touch and pressure improves proprioceptive sensation and contributes to facilitation. Manual contact via touch or pressure is one of the most successful techniques to facilitate motor control, especially when faulty patterns and poor kinesthetic awareness exist. Through facilitation, the physical therapist communicates with the patient using somatosensory cues to foster the desired movement or muscle activity. Firm, deep pressure over the area of the body where a contraction is desired, facilitates that muscle contraction thereby enhancing kinesthetic and sensory awareness. Utilizing sensory cues and the activation of the mechanoreceptors, the patient's attention is directed to the desired movement. In like manner, touch and pressure are proven methods for increasing, or facilitating muscle activation, and stimulation of the skin has been shown to enhance alpha and gamma motor neuron activity in the underlying muscles. These neurons are responsible for producing muscle contraction.

In many instances, however, the use of fingers to stimulate mechanoreceptors is not feasible nor desirable. The exercise being performed may not allow for the physical therapist to maintain contact with the patient or the individual may be performing the exercise or movement outside the presence of their physical therapist. For example, a physical therapist may use their hands or fingers to provide sensory input to the patient while in a static position, such as, lying down. However, if the patient is performing dynamic movements or activity—such as running, walking, weight lifting, cooking, house cleaning, hiking, yoga, playing various sports, strength training, bending, lifting, loading, and the like—the physical therapist cannot use their fingers or hands to apply firm, deep pressure over the area of the body, with respect to the patient, to facilitate proper muscle contraction. In addition, this technique can be effectively applied for improving performance and support, where an individual does not need the assistance of a physical therapist, but would like to improve the way they exercise and/or breathe, improve their performance in a sport or have better stabilization during the activity required by their job. For instance, a runner who needs to improve their trunk stabilization and breathing would not be able to run with their own hands pressing on the required areas of the body where a contraction is desired without the risk of harming themselves. Likewise, it is impractical to have someone running behind them pressing on said areas to stimulate the cutaneous receptors. Consequently, there exists a need for an apparatus for patients to use without the assistance of another human, or their own hands, that increases awareness and sense of the targeted area and facilitates better muscle activity helping them improve or optimize movement and stabilization patterns.

An alternate way of stimulating the mechanoreceptors is by utilizing inwardly facing projections attached to a band, belt, body wrapping, or the like. In such a case, the shape and size of the projections are critical. The pressure the projection applies to the skin, underlying tissues and nerve receptors is what helps to facilitate the muscle activity. The pressure the projection applies to the skin and nerve receptors allows the patient to feel where to contract the muscles, which gives the patient greater ability to contract those muscles and therefore improve the quality of that contraction. If the shape of the projections are too large or pointed said projections can apply too much pressure to the skin and inhibit the patient from being able to contract the target muscles. If said projections are too small or flat, there may not be enough pressure applied or the pressure will be distributed across too large a surface area for the patient to feel, facilitate, and execute the proper muscle contraction.

In light of the above, there exists a need for facilitating proper muscle contractions during any movement or activity, including respiration to provide guidance assuring and improving appropriate contraction patterns.

Further, there exists a need for an apparatus that applies specific pressure to an area of the body to hasten the response of the neuromuscular system through stimulation of the mechanoreceptors and therefore resulting in improved muscle activity and motor patterns. That continues to provide input and feedback throughout the activity.

Still, there exists a need for an apparatus that improves human trunk stabilization such that a person can achieve proper timing and coordination during contraction of the diaphragm, pelvic floor, deep segmental spinal muscles, muscles of the abdominal wall, and the like.

### SUMMARY OF THE INVENTION

The present invention addresses the above stated situation by providing a wearable belt, or body wrapping, for the torso, trunk, or any other muscle, muscle group or area in which a body wrapping can circumscribe, adapted to generate tactile input. The tactile input supplies the cue for the person wearing the belt to contract or tighten into. The belt includes inwardly facing projections which contact abdominal, or other muscles, through the skin.

This replaces the user's fingers or the therapist's fingers. Tactile, or sensory, input arising from the inwardly facing projections are used as cues and provide the user with feedback to improve their sense/awareness of the intended muscle group to be contracted and facilitates improved quality of that contraction. As the user contracts the targeted muscle or performs the activity or movement, sensory feedback occurs as pressure or tightening is increased under the projections and as they are pushed away from the area. In addition, as in the example of using the apparatus to improve trunk stabilization, if the correct activity occurs, there will be an expansion of the abdominal wall into the entire belt. This supplies the user with additional sensory feedback as the skin contacts the belt and as the expansion causes the belt to be stretched there is an increase pressure felt due to the resistance created from the elastic “recoil” of the belt. It is this mechanism of providing both sensory input followed by additional sensory feedback that allows the apparatus to effectively facilitate the proper movement or muscle activity. The sensory input allows the user to have a better understand of and connection to the targeted area, the sensory feedback that follows provides information to the user as to whether the desired muscle activity or movement has occurred. This cycle continues throughout the exercise, movement or activity in order to provide constant facilitation and feedback to the user allowing them to work on maintaining or improving it during the entire duration.

In some implementations, there can be an ability of the inwardly facing projections to generate audible and/or visible advisory, or vibrational outputs if the person is correctly contracting the intended muscles. For example, as the person is able to increase the intensity of the muscle contraction, an audible beep can be heard getting louder as the contraction intensifies. Another implementation can be predetermined intervals of vibratory outputs. For example, vibration signals can be used every 2 minutes as reminders for an individual to actively tighten into the belt when using it for activities of longer duration.

In some implementations, data acquired by transducers incorporated in the projections may be transmitted to mobile communications devices to determine the level of the contraction of the muscle.

In other embodiments, the belt or body wrapping may include, along with the input generating projections, traditional biofeedback mechanisms to help determine if proper coordination/contraction of the muscles is taking place.

In an implementation, the projections which may or may not generate electrical signals may be provided for retrofitting to pre-existing belts.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a perspective view of an apparatus for providing tactile sensible input, according to at least one aspect of the invention;

FIG. 2 is a perspective view of an alternative apparatus with a first and second removably extendable attachment member for providing tactile sensible input, according to a further aspect of the invention;

FIG. 3A is a mobile communications device, which may both receive and send data to the belt of FIG. 1 utilizing a wireless signal or a connection to a network;

FIG. 3B is an electrical schematic of circuitry which may be associated with the belt of FIG. 1;

FIG. 4 is a perspective view of an apparatus for providing tactile sensible input, according to at least one aspect of the invention, where the plurality of projections are encased in pouches;

FIG. 5 is an interior view of an apparatus for providing tactile sensible input, according to at least one aspect of the invention;

FIG. 6 is a perspective view of a projection encased in a pouch for providing tactile sensible input, according to at least one aspect of the invention, where the pouch may attach to a belt utilizing loops;

FIG. 7 is an exterior view of an apparatus for providing tactile sensible input, according to at least one aspect of the invention, where the projections are removably affixed to the belt via loops;

FIG. 8 is an exterior view of an apparatus for providing tactile sensible input, according to at least one aspect of the invention, where the secondary exterior pressure enhancing band is illustrated therein;

FIG. 9 is an interior view of an apparatus for providing tactile sensible input, according to at least one aspect of the invention where the secondary interior pressure enhancing band is illustrated therein;

FIG. 10 is a perspective view of an apparatus for providing tactile sensible input, according to at least one embodiment of the invention;

FIG. 11 is a rear view of an apparatus for providing tactile sensible input, according to at least one aspect of the invention, that illustrated removably attachable shoulder straps are further depicted;

FIG. 12 is a perspective view of an apparatus to be worn around the pelvis or the lower rib cage for providing tactile sensible input, according to at least one embodiment of the present invention;

FIG. 13 is a perspective view of a projection for providing sensible input, according to a further aspect of the invention, where the projections may include a material with a higher coefficient of friction on the top surface area;

FIG. 14 is an illustrative diagram which may be associated with the belt of FIG. 1 for communicating to the user an embodiment signaling to the user, or communicating via a wired or wireless network means to an external device that may signal to the user;

FIG. 15 is a block diagram of how to use the apparatus for general weight lifting and exercises;

FIG. 16 is a block diagram of how to use the apparatus for weight lifting and exercises;

FIG. 17 is a block diagram of how to use the apparatus for weight lifting and exercises in conjunction with electronic sensors;

FIG. 18 is a block diagram of how to use the apparatus for back support;

FIG. 18A is a block diagram of how to use the apparatus for back support;

FIG. 18B is a block diagram of how to use the apparatus for back support;

FIG. 18C is a block diagram of how to use the apparatus for back support;

FIG. 19 is a block diagram of how to use the apparatus for back support in conjunction with electronic sensors;

FIG. 19A is a block diagram of how to use the apparatus for back support in conjunction with electronic sensors;

FIG. 19B is a block diagram of how to use the apparatus for back support in conjunction with electronic sensors;

FIG. 19C is a block diagram of how to use the apparatus for back support in conjunction with electronic sensors;

FIG. 20 is a block diagram of how to use the apparatus for respiration training.

FIG. 21 is a block diagram of how to use the apparatus for targeting muscles and other areas other than core muscles;

FIG. 22 is a block diagram of how to use the apparatus according to at least one aspect of the invention; and,

FIG. 23 is an illustration of an apparatus that provides tactile sensible input to the user, according to at least one aspect of the invention, while providing biofeedback to the user in response to muscle movements performed.

### DETAILED DESCRIPTION

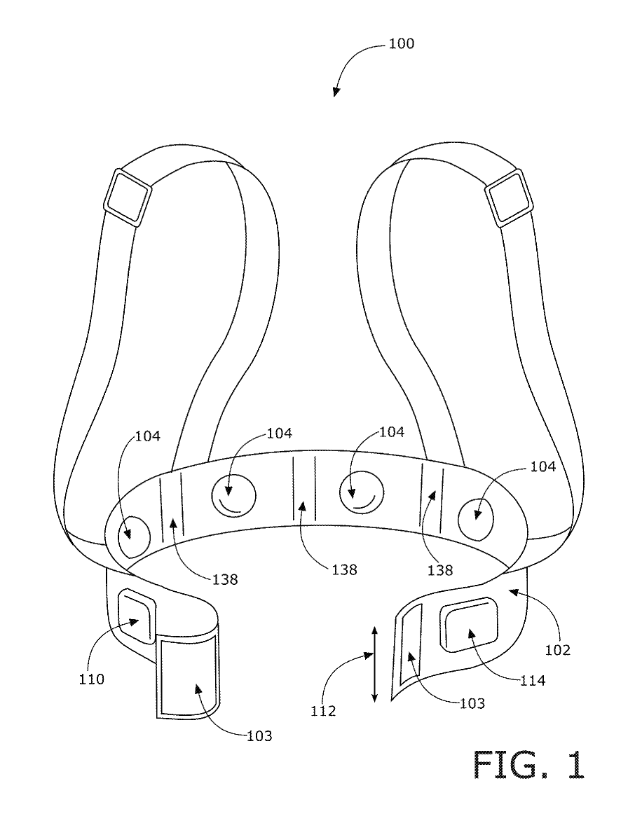

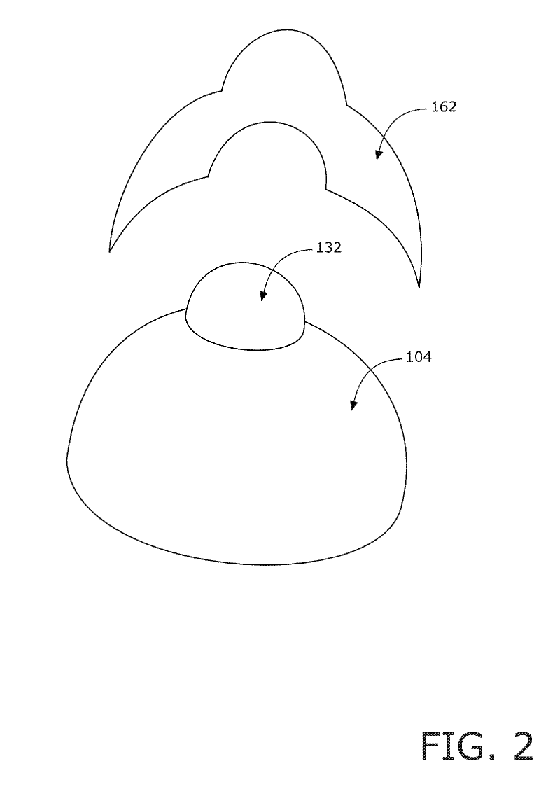

Referring first to FIG. 1, according to at least one aspect of the invention, there is shown an apparatus 100 for providing sensible/tactile input to enable a person to control and improve contraction of muscles during daily activities, including exercising, by stimulating the mechanoreceptors with respect to a specific muscle or muscle group. The apparatus 100 is associated with a belt 102 (in the present disclosure “belt” and “body wrapping” are interchangeable) worn around the body (not shown). The apparatus 100 includes a plurality of projections 104 associated with the belt 102. The projections 104 may each be blunt, for example, being substantially semispherical as depicted, spheroid or other. In some preferred embodiments, the projections are 2.5″ in maximum diameter domes (also referred to as semi-spherical projections), however, the range of the dome maximum diameter can range from 0.25″ to 5.0″ and still be effective in providing tactile input for certain users. The projections 104 may be substantially semi-spherical having an x-axis defining a diameter and a y-axis defining a radius wherein the radius is 15% to 85% of the diameter. Moreover, the projections 104 may each comprise a single resilient or elastic member such as rubber or relatively dense foam. Essentially any material that can be formed substantially into a half dome and that can provide a focused area of pressure without collapsing when attached to the belt and worn by the user is feasible. As discussed more fully below, the projections may comprise one or more pressure, transducers or EMG sensors. Further, the projections 104 may utilize a plurality of removably extendable attachment members 132, 162 (FIG. 2), that are also substantially semi-spherical or dome-shaped, which can be mounted on larger projections to create a nipple-like focal point to facilitate more precise contact with the skin to facilitate mechanoreceptor stimulation. The plurality of removably extendable attachment members 132, 162 may each be blunt, for example, being substantially semispherical as depicted, spheroid or other. Further, in a preferred embodiment, the plurality of removably extendable attachment members 132, 162 may each comprise a single resilient or elastic member such as rubber or relatively dense foam. Essentially any material that can be formed substantially into a half dome and that can provide a focused area of pressure without collapsing when attached to the belt and worn by the user is feasible.

In these nipple-like embodiments, the diameter of the dome is much smaller (0.25″ to 1.5″ maximum diameter) to provide the desired more focused tactile input when worn by the user. The nipple-like projections may be attached to the larger projections 104 by any common attachment method. It is understood that the profile of the projection plus nipple attachment could be molded as one piece rather than two as shown in the figures. It is to be understood that other shapes and sizes may be desirable depending on the application and the user's sensitivity to the tactile input the projections provide.

The belt 102 may be elastic and resilient, having a width 112 of about 2″ to 8″, and sufficient length to entirely encircle the body. That part of the body which may be encircled may be the arm, leg, trunk, or hip. The belt 102 may encircle the abdominal area for facilitation, at any point from just below the ilium up to the lowermost three or four non-floating ribs. Alternatively, the belt 102 may be placed elsewhere for facilitating, for example, ribs, thoracic spine, the gluteal muscles, trapezius muscles, the quadriceps muscles, and the medial gastrocnemius. The length of the belt 102 will be determined accordingly, and may be adjustable if desired to accommodate encircling all of the possible parts of the body where there is a need to improve muscle contraction or movement. Fastening and adjustment of length of the belt 102 may be performed by hook and loop fastener 103, or by any other suitable arrangement of fasteners.

The above described belt 102 and accompanying features is effective in providing the tactile input to a user prior to contraction. These features may be supplemented with the incorporation of various electronic features that will be described more fully below.

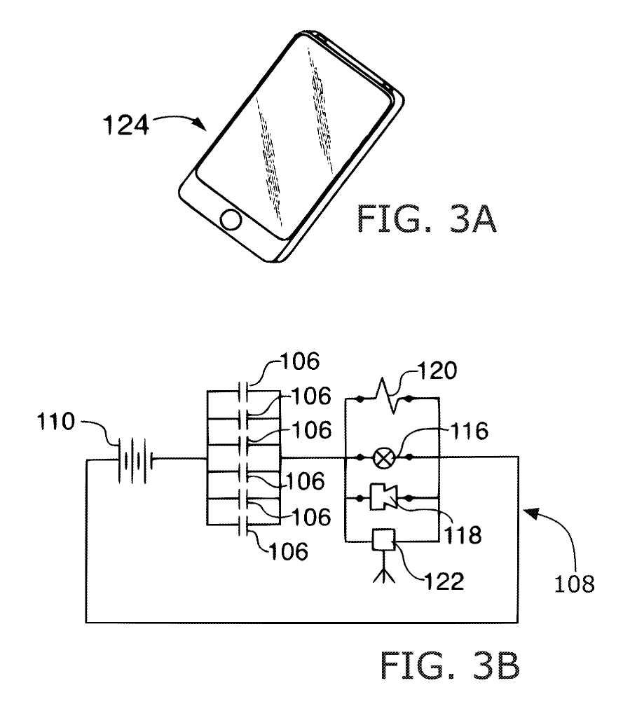

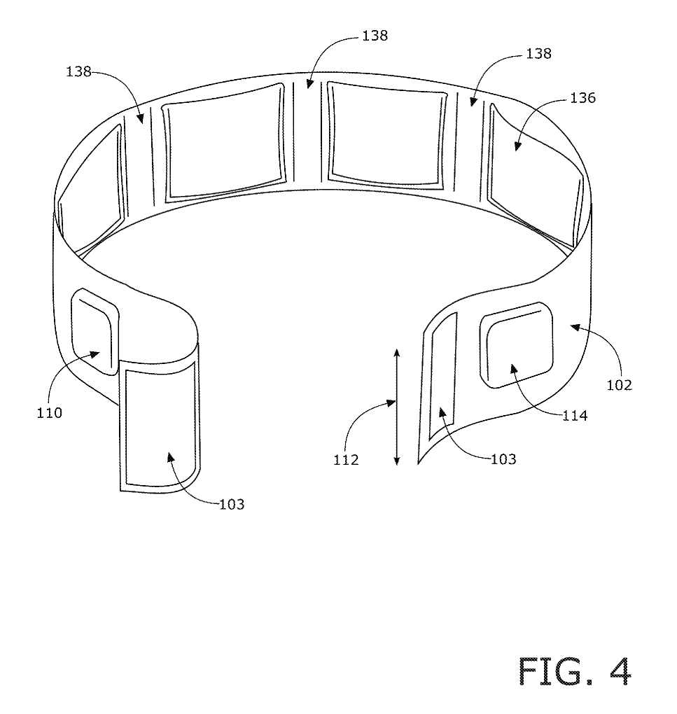

In an alternative embodiment, each one of the projections 104 may include a transducer 106 (FIG. 3, see also FIG. 4) arranged to generate a signal when the muscles contract. In this embodiment the apparatus 100 includes an electrically powered signaling device 114, and a power circuit 108 (FIG. 3) including a power source 110 operably connecting each one of the projections 104 to the electrically powered signaling device 106. Electrical conductors of the power circuit 108 may be protectively contained and concealed within the belt 102. As depicted, the transducer 106 may comprise a pair of normally open contacts which close when a predetermined level of pressure is exerted on the projections by distension of the muscles. Alternatively, the transducer 106 may comprise a piezoelectric device, a pneumatic or hydraulic bulb arranged to transmit a pressure signal (none of these options are shown but are well known in the art). Further the belt may include paraspinal muscle sensors 156 which would contact the paraspinal muscles on each side of the spine when the belt is worn by a user (FIG. 4). Again, in a manner similar to the transducers 106 place on the ends of the projections 104, a user would be alerted by sound, light, etc. when the paraspinal muscles are activated during a certain physical activity.

Paraspinal sensors would likely have its primary role in the rehab setting where healthcare practitioners would be working with individuals who have experienced low back pain or are post-operative lumbar spine surgery. It would also provide better feedback once these individuals continue to work on improving their stabilization function and movement on their own. It is common in our society, especially with those experiencing low back problems for people to over use their lumbar paraspinal muscles as spine or trunk stabilizers. These individuals may be able to contract their abdominal muscles with some expansion, but may still have poor timing and be excessively contracting their paraspinal muscles. This creates overload on the spine and its structures and can cause continued low back issues, dysfunction and injury. It is important for people to be able to properly contract their spinal stabilizers independently of excessive and early lumbar paraspinal muscle activity—which should be the last muscle group to contract at the appropriate level, when trunk stabilization is needed. When using the belt with projections only, it may appear to the healthcare practitioner and/or the user that they are performing the correct contraction by noting the abdominal wall expanding into the belt and the projections pushing out and moving away from the trunk. But what it doesn't tell them is if the user is also contracting their paraspinal muscles.

As shown in FIG. 4, the sensors would be placed along the paraspinal muscles and could be permanently implanted in the belt 102 or as a separate piece that wraps around the belt in order to be adjusted to accommodate for individual anatomy. Electrodes are flat in composition, because they are meant only to pick up muscle activity, so they would not serve to facilitate the muscle activity of the paraspinals, which is not desired. But their purpose would be to provide the user with information about when and to what extent their paraspinal muscles are contracting. They would serve a “negative” feedback function. Whether there was a read out on a phone screen or just an increasing beeping sound, as the user attempted to use the belt, if they were also contracting their paraspinal muscles, they would hear a beep that started low with light activity and increased in pitch as the muscle activity increased. The goal would vary depending on initial level of impairment (i.e. how poorly they were activating their muscles) but would be improving contraction of the abdominals and associated core muscles while limiting excessive paraspinal activity.

At this stage it is important to distinguish once again that the transducers and paraspinal muscle sensors 156 are biofeedback mechanisms that are used as an adjunct to aid a user or a physical therapist working with a client as to whether the tactile input created by the projections are creating the desired coordinated contraction. This is a completely different than devices that are purely biofeedback devices as described in the Description of the Prior Art section above. Most important is the tactile input that the belt 102 creates prior to contraction, and the sensory feedback it creates when proper contraction or movement has been accomplished

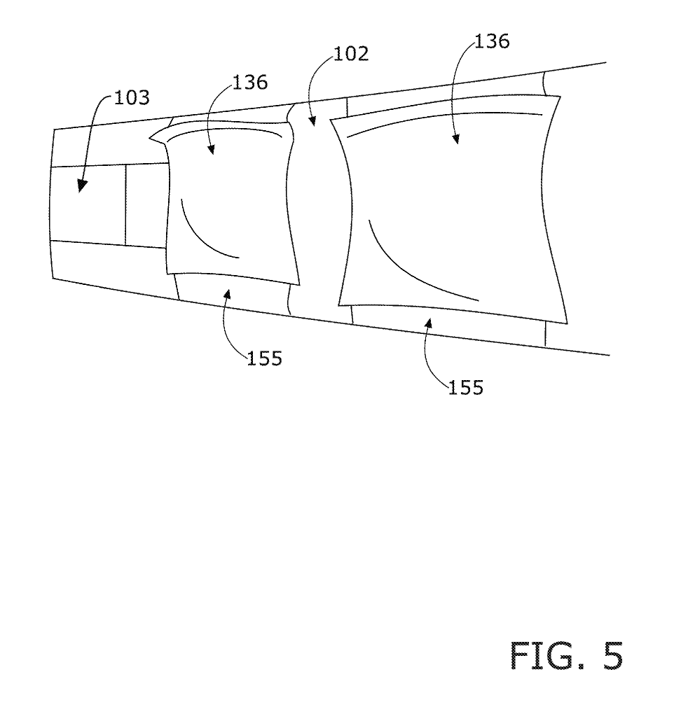

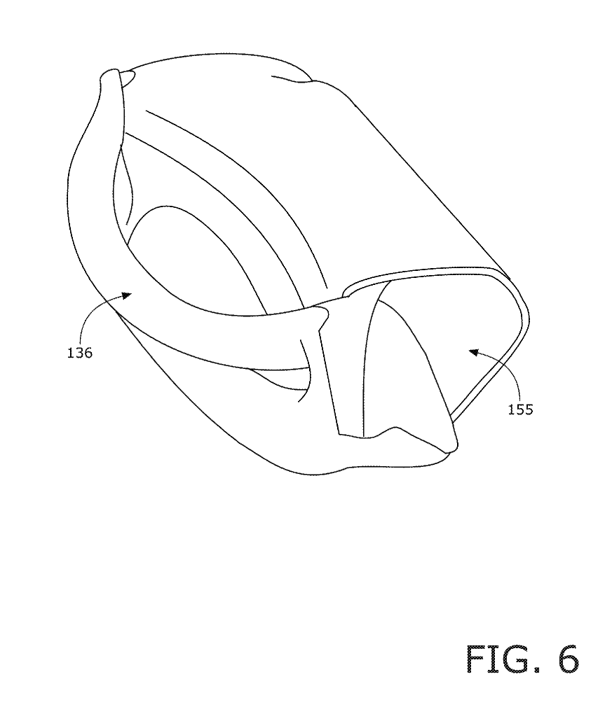

In either case when the belt 102 includes projections which may or may not include a transducer, the projections 104 may be detachably fixed to the belt 102 by hook and loop material or by any suitable fastener. The projections 104 may be movably secured to the belt 102 via loops 155 and can slide therealong (FIG. 5) to properly position the projections and/or held in pouches or pockets 136 (see FIGS. 5 and 6) that are also slideably attached along the length of the belt 102 via loops 152. Regardless of the nature of the mounting of the projections 104, they may be adjustably positioned along the belt 102 to achieve optimum response of the muscle activity and if present, the transducers 106, allowing for best accommodation to different body dimensions and configurations given individual anatomical variation. It is to be understood that both the loop 155 and pouches 136 may be elastic and resilient or comprised of any suitable material having elastic and resilient properties and are secured to the belt 102 by hook and loop material or by any suitable fastener (FIG. 5). The elasticity of the belt 102 is a key feature of the instant invention and may encompass other suitable materials that provide the property of a material that enables it to change its length, volume, or shape in direct response to a force effecting such a change and to recover its original form upon the removal of the force.

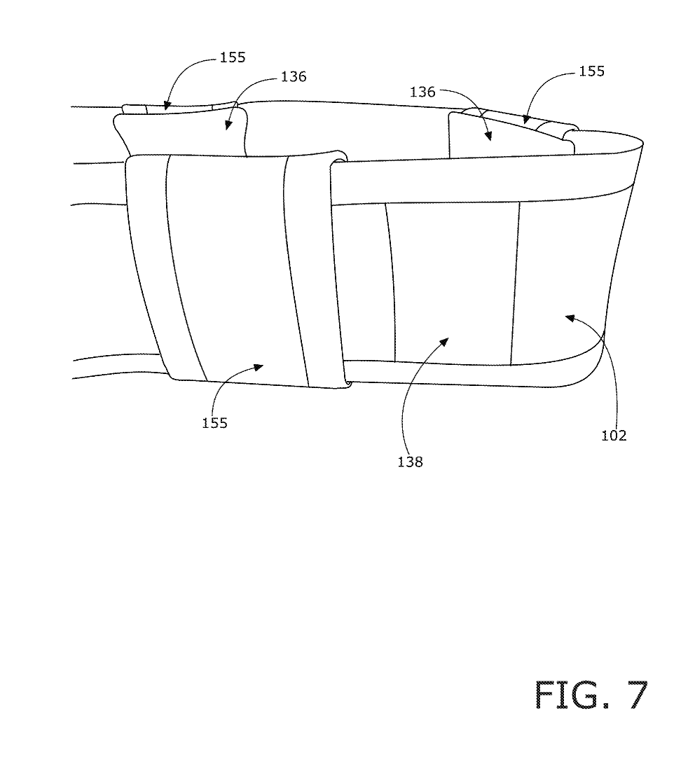

In addition, the belt 102 may also utilize a plurality vertical stays 138 (FIG. 1, FIG. 7) that are removably and adjustably retained within or on the belt and further adapted to maintain varying levels rigidity to provide additional support for the belt 102 and/or the user and are comprised of silicone or a firm foam or the like. Moreover, the vertical stays 138 may be comprised of an additional layer of elastic placed at key areas of the belt 102 where increased vertical compression results, for example, a user bending at the waist. Additionally, the vertical stays 138 give the belt 102 resistance to the vertical compression and prevent buckling of the belt 102 at those areas. Moreover, the vertical stays keep the projections 104 from flipping or rotating during use. When flipping or rotating of the projections 104 occur, the belt 102 is far less effective and becomes cumbersome.

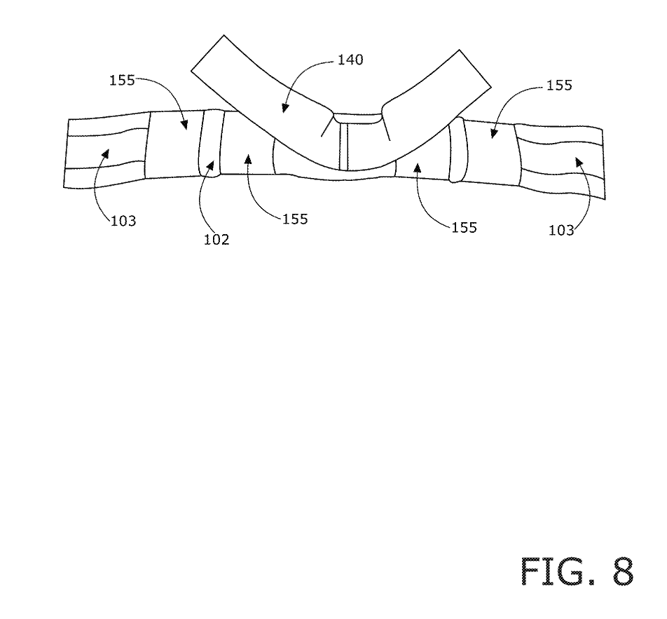

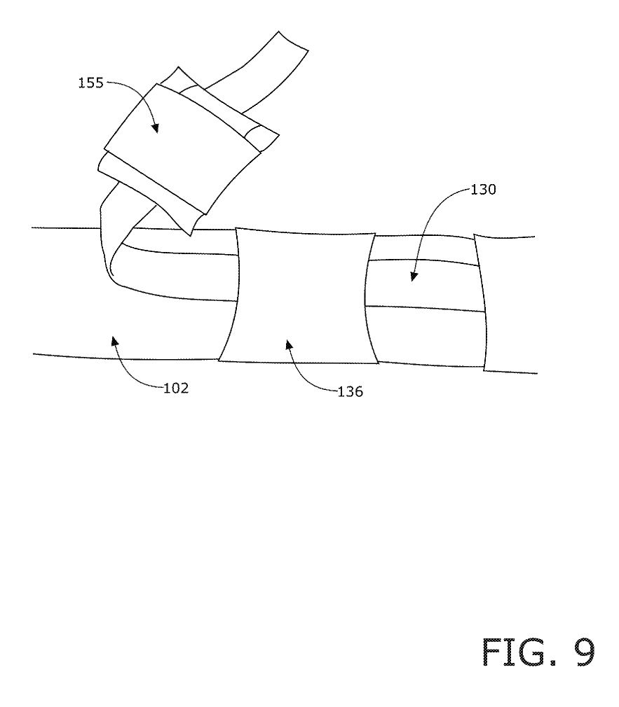

Turning to FIG. 8, an exterior view of the belt 102 is illustrated. A secondary exterior force enhancing band 140, of different elastomeric composition of the belt 102, is attached to the mid portion of the belt 102 as shown and is used to encircle the exterior of the belt 102. In a preferred embodiment, the purpose of the secondary exterior pressure enhancing band 140 is to prevent flipping or sliding of the projections 104 during extreme movements, which is further operative to be removably affixed to the belt 102 and can be used either as an attachment to the belt 102 or by itself. Moreover, the secondary exterior pressure enhancing band 140 may also bear protrusions 104, in the same manner as the belt 102. The secondary exterior pressure enhancing band 140 is utilized to provide additional pressure upon the projections 104 when the belt 102 encircles a part the body, such as, the arm or leg, the trunk, hips, and the like. In a preferred embodiment, the purpose of the secondary exterior pressure enhancing band 140 is to prevent flipping or sliding of the projections 104 during extreme movements. The additional pressure increases the degree to which projections, with or without attachably releasable attachment members 132, are sensed/felt. Alternatively, a secondary interior pressure enhancing band 130 may be used in conjunction with the interior surface of the belt 102 for the same benefit and purpose of the secondary exterior pressure enhancing band 140 as described above. See FIG. 9.

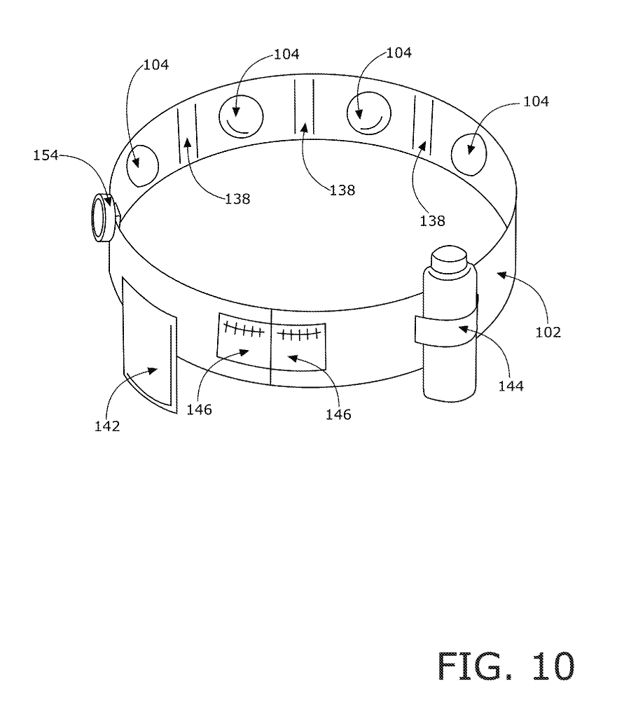

In an alternate embodiment, FIG. 10, one or more phone holders 142, one or more drink holders 144, one or more pockets with or without a zipper 146, and one or more light emitting components 154 may be removably secured to the belt 102 by hook and loop fasteners 103, or by any other suitable arrangement of fasteners, such as clips, hooks, magnets, loops 155 and the like.

The projections 104 may be provided in different dimensions and different degrees of resistance to compression in order to accommodate the sensitivity and anatomical variations of the individual person. The degree of resistance to compression, which may be determined, e.g., by projections 104 of different elastomeric composition or pressure sensors/transducers of different spring or density characteristics, will be perceived by the user as variable firmness.

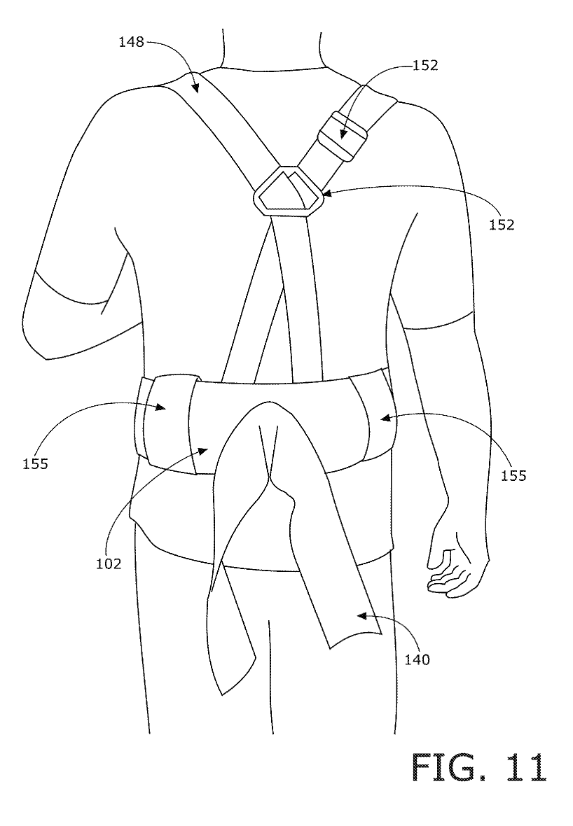

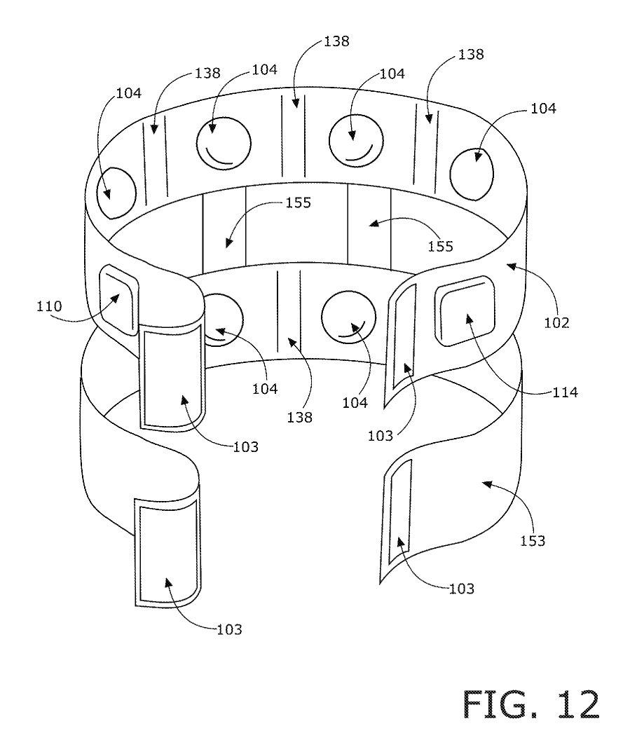

Referring to FIG. 11, it is also contemplated that the belt 102 may include detachable straps 148, adjustable by strap adjusters 152 or the like, that can be used to maintain the belt about the body when not fully engaged. For example, back support belts, as used by various personnel during lifting activities, often include shoulder straps that are used to maintain the belt upon the body when the support belt is not engaged around the trunk.

FIG. 6 illustrates an embodiment in which the projections 104 are encased in sleeves 137 that include pouches 136 and are slideably attached to the belt 102 by the use of loops 155 (see also FIG. 7). As mentioned above, the pouches 136 may be retrofitted to pre-existing belts. Similarly, FIG. 7 illustrates an exterior view of the belt 102, according to at least one aspect of the invention, for providing tactile input whereby the projections (not shown) are protected and enveloped by a plurality of pouches 136 and slideably attached to the belt 102 via attachment loops 155. As illustrated, a plurality of vertical stays 138 section the belt 102. The vertical stays 138 are removably and adjustably supported and further adapted to maintain varying levels rigidity to provide additional support for the belt 102 and are comprised of soft silicone or a firm foam or the like. Moreover, the vertical stays 138 may be comprised of an additional layer of elastic placed at key areas of the belt 102 where increased vertical compression results, for example, a user bending at the waist. Additionally, the vertical stays 138 give the belt 102 resistance to the vertical compression and prevent buckling of the belt 102 at those areas. Moreover, the vertical stays keep the projections 102 from flipping or rotating during use, as described above.

FIG. 12 illustrates an alternate embodiment where a supplemental stimulation band 153 is removably secured to the belt 102 to add stimulation to additional areas concurrently with the said belt 102. The supplemental stimulation band 153 may utilize a plurality of attachment loops 155 that would slideably attach over the main belt 102. The supplemental stimulation band 153 may have additional projections 104 that may or may not be encased in pouches. The supplemental stimulation band 153, in conjunction with the belt 102, may encircle the pelvis or lower rib cage of the user (not shown). Further, the supplemental stimulation band 153 may be used for both trunk stabilization and respiration retraining. A patient who lacks proper respiration, trunk stabilization, or both, can use additional facilitation when they are first learning how to correctly perform the desired movement/action/etc.

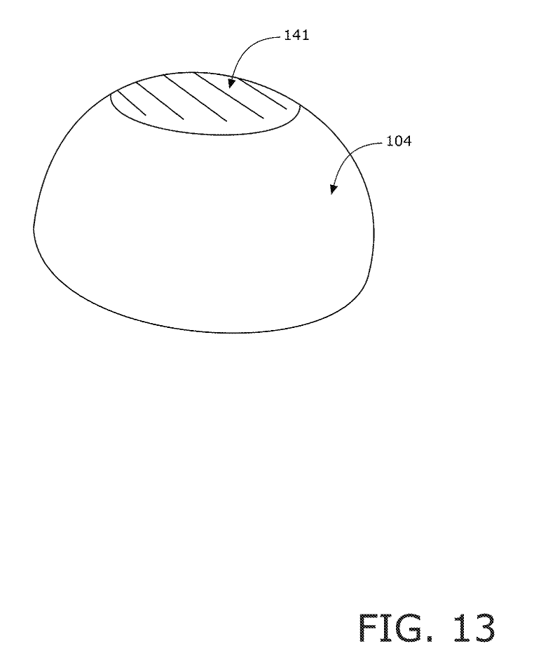

In yet another embodiment, as shown in FIG. 13, the projections 104 may include a material with a higher coefficient of friction on the top surface area 141 of the projections 104 to assist the projections 104 and the belt 102 to remain in place during physical activity.

Referring particularly now to FIG. 3, and referring back to the electronically assisted belt 102, an electrically powered signaling device 114 may be an indicating lamp 116 which produces a visible signal, a buzzer 118 or another type of sound producing device which produces an audible signal, a vibratory device comprising for example an oscillating solenoid 120 which produces a vibratory signal, or a radio frequency transmitter 122. In certain embodiments, each projection 104 may be equipped with the necessary means to provide a buzzer 118, or vibrating sensation, and/or audible signal to be triggered at various thresholds with regards to a pressure sensor 172. While this could be associated with the mobile communications device 124, each projection 104, respectively, may so include an on/off switch or dial controlling the frequency of such vibrating or audible signals to the user; a dial to control the frequency of said vibrating or audible signals to the user; and/or a dial/switch to select either an audible signal, vibratory signal, or both.

Because the belt 102 is an active retraining device, it is not intended to be put on and forgotten about. It is intended to be used consciously by the user. As such, when using the belt 102 for longer term activities; such as running, hiking, tennis, yoga, lifting, and the like, it may become important for the user to be reminded to use the belt 102 to contract their muscles in the proper manner appropriate for the activity due to the potential effect of habituation and desensitization of the tactile input provided by the projections 104. For example, the projections 104 may vibrate and/or create audible signals at specified intervals, e.g., every 2 minutes; every 5 minutes; and the like to remind the user and cue them to refocus on the tactile input being provided by the projections. To that end, each projection 104 may so be equipped with the appropriate dials, buttons, switches, and or triggers for the user to set the time interval of the desired signals. It should be noted that the vibratory signals may also serve as additional sensory input along a different nerve path in order to help the user better feel the muscle or muscle group in which the mechanoreceptors require active stimulation by the projections 104.

The electrically powered signaling device 114 may include more than one of the electrically powered signaling devices 114, including any combination of the listed types of electrically powered signaling devices 114.

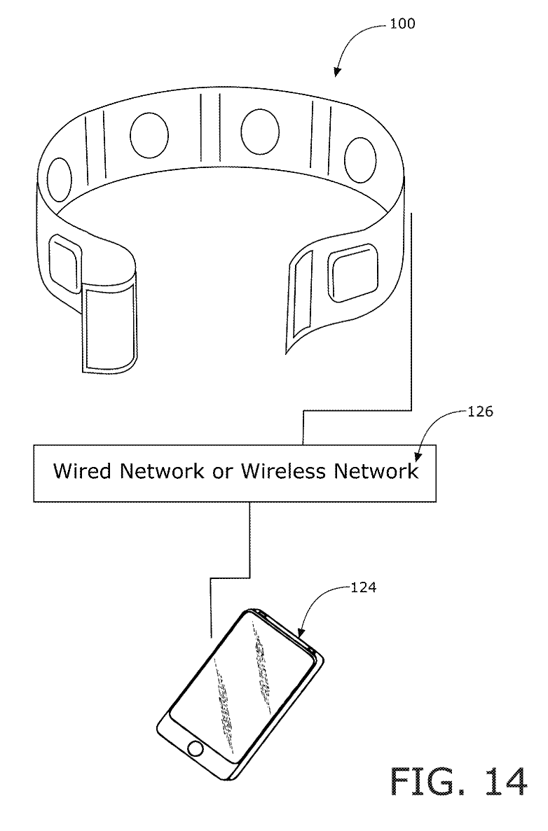

Turning again to FIG. 3, pursuant to at least one aspect of the invention, the electrically powered signaling device 114 including the radio frequency transmitter 122 may operate in conjunction with a mobile communications device 124. The radio frequency transmitter 122 is in communication with each one of the transducers 106, being energized for example by closure of any one or more of the normally open contacts of the transducers 106. The radio frequency transmitter 122 operates at a frequency to which the mobile communications device 124 is responsive. The specific type of mobile communications device 124 will be understood to include a data processor, a display in communication with the data processor, a memory in communication with the data processor, and computer instructions loaded into the memory and operable to generate an output on the display which output is indicative of at least one muscle actuating at least one of the transducers 106. As such, the mobile communications device 124 (FIG. 14) may include virtually any device capable of receiving and sending a message over a network, such as network 126 (FIG. 14), or the like. Such devices include portable devices such as, cellular telephones, smart phones, display pagers, radio frequency (RF) devices, music players, digital cameras, infrared (IR) devices, Personal Digital Assistants (PDAs), handheld computers, laptop computers, wearable computers, tablet computers, integrated devices combining one or more of the preceding devices, or the like. For example, an iPhone® may be utilized as the mobile communications device 124, when a suitable application has been downloaded thereonto. The application drives the display of the iPhone responsively to receiving a radio frequency signal ultimately generated by closure of the normally open contacts of at least one transducer 106.

Mobile communications device 124 also may include at least one other client application that is configured to receive content from another computing device, including, without limit, server computing devices (not shown). The client application may include a capability to provide and receive textual content, multimedia information, or the like. The client application may further provide information that identifies itself, including a type, capability, name, or the like.

Referring particularly to FIG. 14, the mobile communications device 124 may be connected to the apparatus 100 via a wired or wireless network 126. The network 126 may be configured to couple mobile communications device 124 to apparatus 100. Network 126 may include any of a variety of wireless sub-networks that may further overlay stand-alone ad-hoc networks, and the like, to provide an infrastructure-oriented connection for mobile communications device 124. Such sub-networks may include mesh networks, Wireless LAN (WLAN) networks, cellular networks, and the like. Network 126 may further include an autonomous system of terminals, gateways, routers, and the like connected by wireless radio links, and the like. These connectors may be configured to move freely and randomly and organize themselves arbitrarily, such that the topology of network 126 may change rapidly.

Likewise, network 126 may further employ a plurality of access technologies including 2nd (2G), 3rd (3G) generation radio access for cellular systems, WLAN, Wireless Router (WR) mesh, and the like. Access technologies such as 2G, 3G, and future access networks may enable wide area coverage for mobile devices, such as mobile communications device 124 with various degrees of mobility. For example, network 126 may enable a radio connection through a radio network access such as Global System for Mobil communication (GSM), General Packet Radio Services (GPRS), Enhanced Data GSM Environment (EDGE), WEDGE, Bluetooth, High Speed Downlink Packet Access (HSDPA), Universal Mobile Telecommunications System (UMTS), Wi-Fi, Zigbee, Wideband Code Division Multiple Access (WCDMA), and the like. In essence, network 126 may include virtually any wireless communication mechanism by which information may travel between mobile communications device 124 and the apparatus 100, network, and the like.

Communication mechanisms within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communications links known to those skilled in the art. Furthermore, remote computers and other related electronic devices could be remotely connected to either LANs or WANs via a modem and temporary telephone link. Network 106 may include any communication method by which information may travel between computing devices. Additionally, communication media typically may enable transmission of computer-readable instructions, data structures, program modules, or other types of content, virtually without limit By way of example, communication media includes wired media such as twisted pair, coaxial cable, fiber optics, wave guides, and other wired media and wireless media such as acoustic, RF, infrared, Bluetooth, and other wireless media.

Any of the above-mentioned electrically powered signaling devices 114 may be separate from the belt 102, as occurs with the mobile communications device 124. For example, the buzzer 118 or the oscillating solenoid 120 may be arranged for mounting on the head or arm of the user. Alternatively, the buzzer 118 or the oscillating solenoid 120 may be arranged for mounting on the head 106 of the projections 104 or within each projection 104 respectively (see FIG. 4). To accommodate this option, the apparatus 100 is provided with suitable signaling apparatus, such as using the Bluetooth protocol or any other low powered system (none shown), and separate power sources 110 distributed among the separated components, where necessary.

The apparatus 100 operates by providing tactile input to certain muscles which generates a signal to the brain improving the connection between the two and facilitating the contraction of the specific muscle and/or muscle groups. This tactile input enables the user to discern the particular muscles which are to be contracted, and in the course of dynamic or ongoing movement, improve the timing, coordination and effectiveness of the muscular contractions. This timing and coordination of contractions may be compared to predetermined ideal norms which have been established as desirable, and allow the person to make appropriate changes when required.

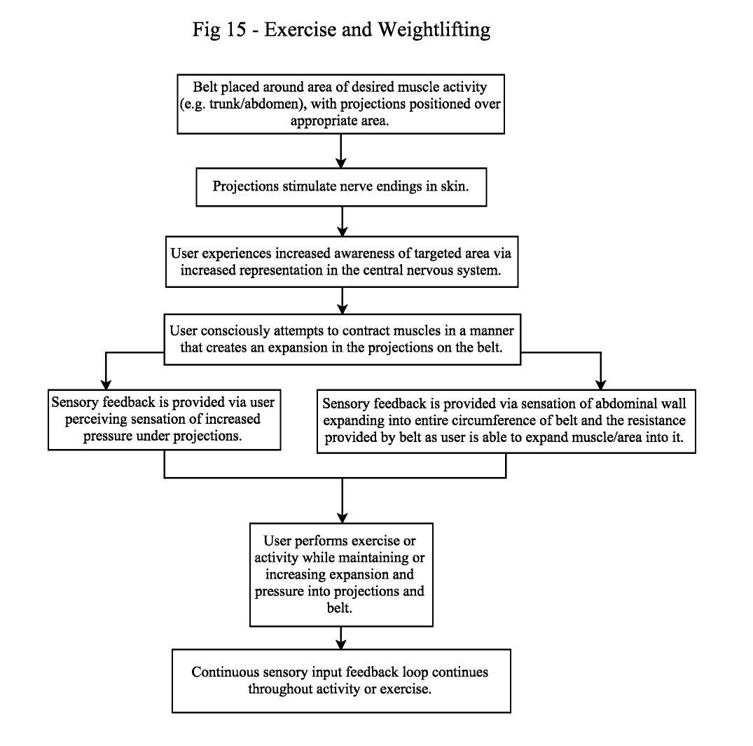

Referring to FIG. 15, according to at least one aspect of the present invention, the invention may be thought of as a method 200 of increasing kinesthetic awareness of an insufficient muscle, muscle group or area for the purpose of facilitating and improving coordinated muscular activity necessary for all purposeful movement, especially those necessary for proper trunk stabilization. The method 200 includes a step of providing at least one inwardly facing projection 104 on a body wrapping (such as the belt 102) placed over muscles being contracted in the course of exercising (block 202); discerning tactile input from at least one of the inwardly facing projections upon at least one muscle (block 204); and contracting said muscle upon which the tactile input from the inwardly facing projection is provided (block 205). The method 200 may also include comparing at least one of identity of a contracting muscle or muscle contraction with a predetermined standard of identity of the contracting muscle or of muscle contraction known to be appropriate for the exercise (block 206). If the muscle contractions are in accord with their predetermined counterparts, or standards, the method 200 comprises continuing the exercise unmodified if the identified contracting muscle is the same as the predetermined muscle contraction (block 208). If, however, if the identified contracting muscle is different from the predetermined muscle contraction, the method 200 comprises modifying an aspect of the exercise until the standard or predetermined muscle contraction are matched (block 210).

In the method 200, providing at least one inwardly facing projection on the body wrapping may include providing the belt 102 on which the at least one inwardly facing projection 104 is mounted (block 212). The method 200 may further include placing the belt 102 around body musculature with the at least one inwardly facing projection 104 facing inwardly, toward the body musculature (block 214).

Because different parts of the body will benefit from the input provided by the present invention for better coordinated muscle contraction, placing the belt 102 around body musculature may comprise placing the belt 102 around the abdomen (block 216); or alternatively, placing the belt 102 around body musculature may comprise placing the belt 102 around the body below the abdomen (block 218).

The method 200 may comprise generating electrical signals responsively to contacting contracted muscles, and using the signals to generate at least one of an audible signal indicative of that muscle contacting the belt 102, a visible signal indicative of that muscle contacting the belt 102, and a vibratory signal indicative of that muscle contacting the belt 102 (block 220).

The method 200 may comprise displaying signals indicative of the muscle contacting the belt 102 on the mobile communications device 124 (block 222).

Further modes of use of the above described device are presented in the attached flow charts. A summary of each flow chart is provided below.

FIG. 15 is a block diagram of how to use the apparatus for general weight lifting and exercises;

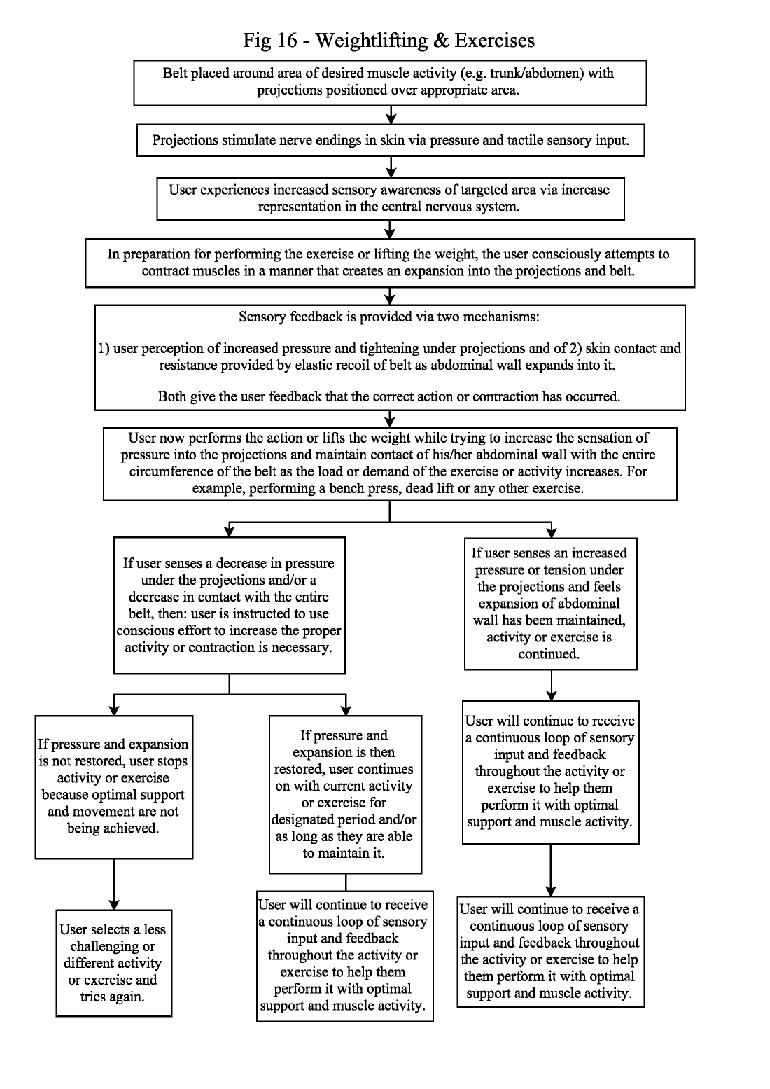

FIG. 16 is a block diagram of how to use the apparatus for weight lifting and exercises;

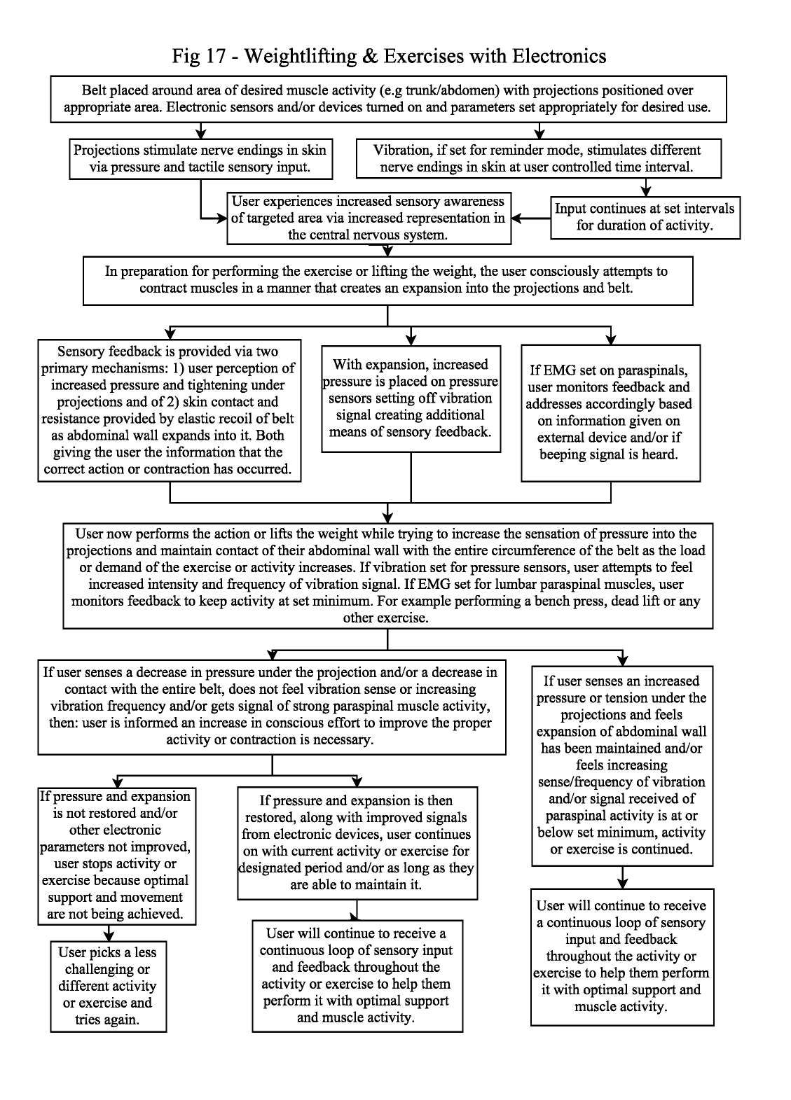

FIG. 17 is a block diagram of how to use the apparatus for weight lifting and exercises in conjunction with electronic sensors;

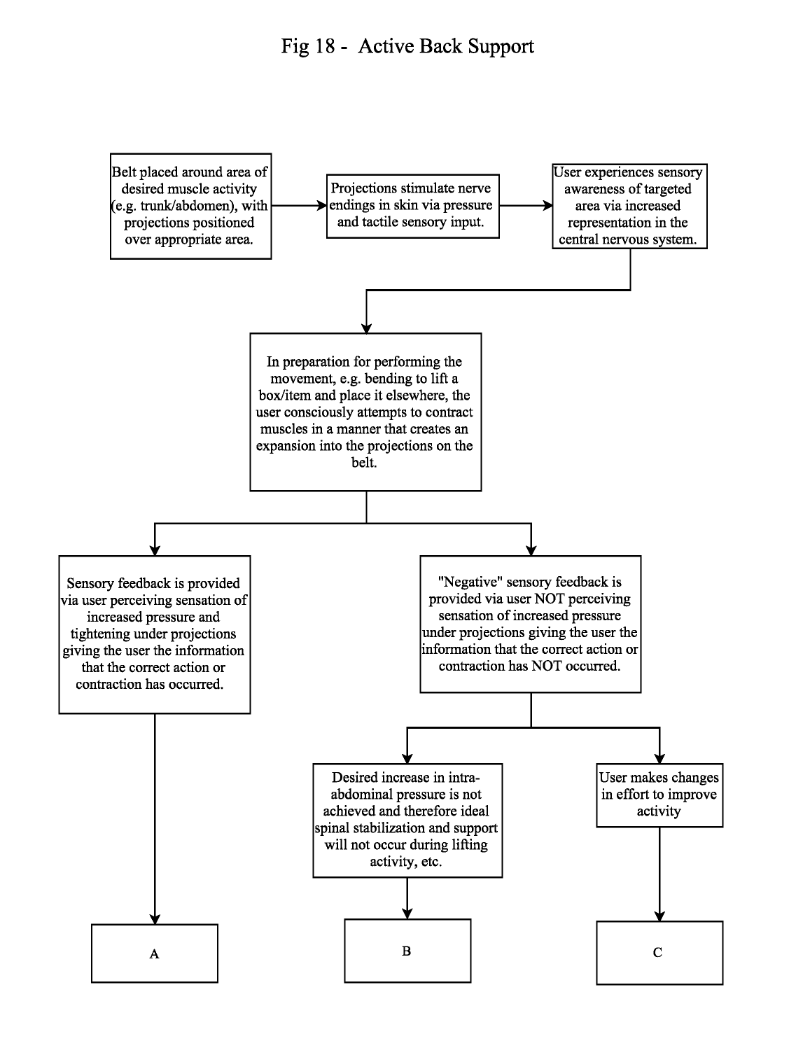

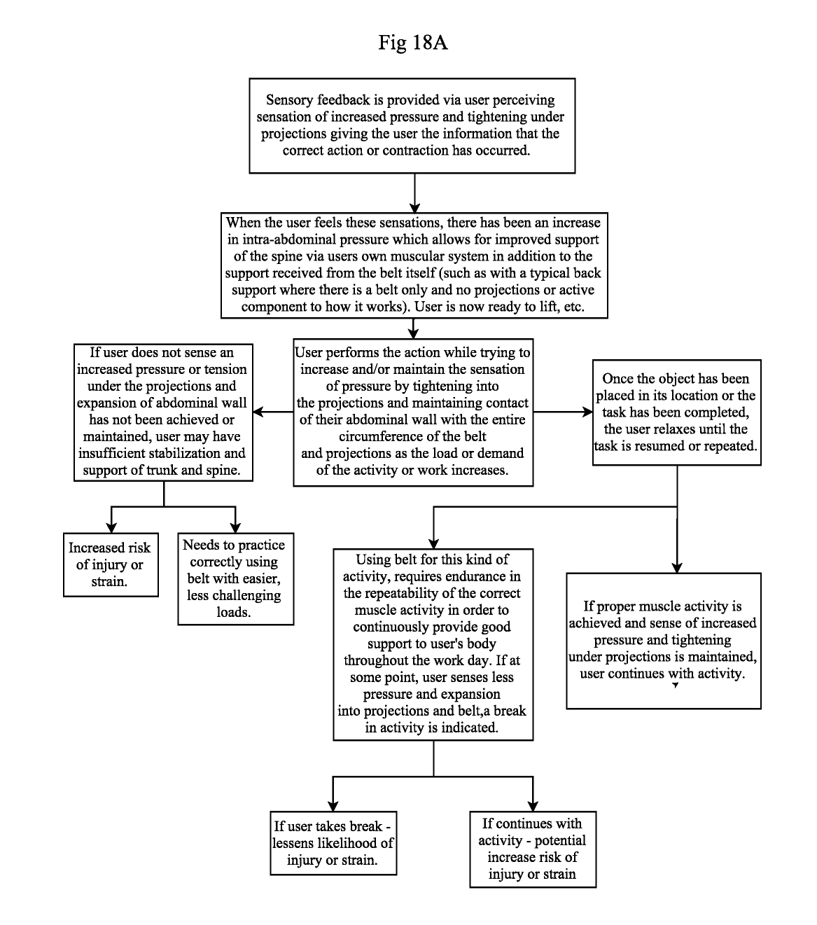

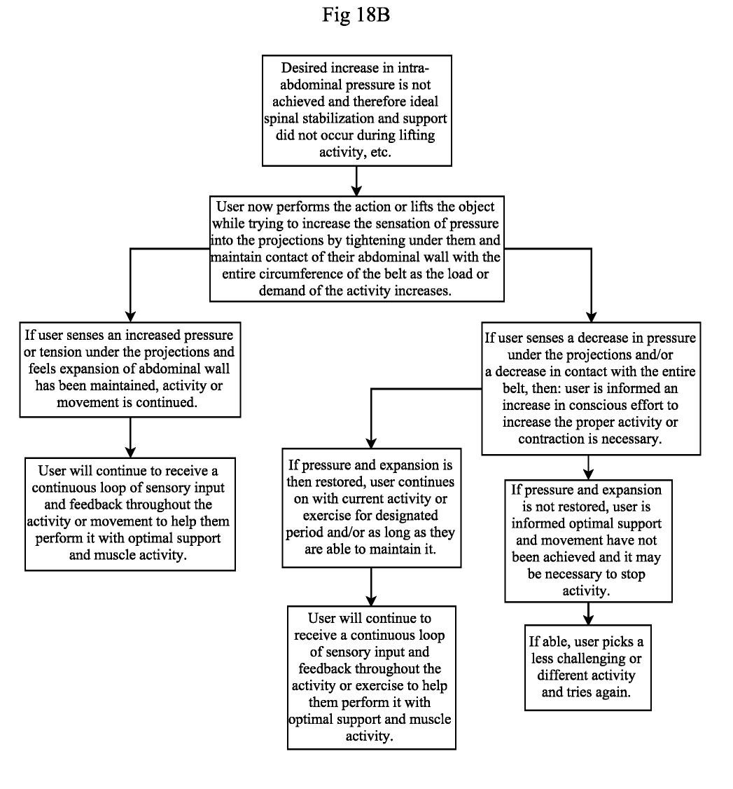

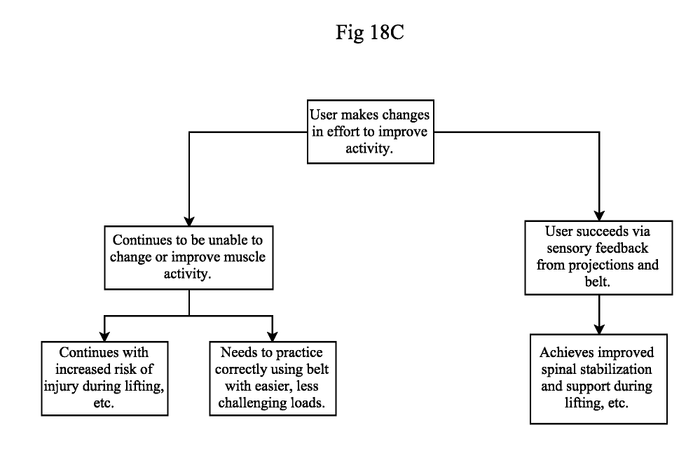

FIG. 18 is a block diagram of how to use the apparatus for back support;

FIG. 18A is a block diagram of how to use the apparatus for back support;

FIG. 18B is a block diagram of how to use the apparatus for back support;

FIG. 18C is a block diagram of how to use the apparatus for back support;

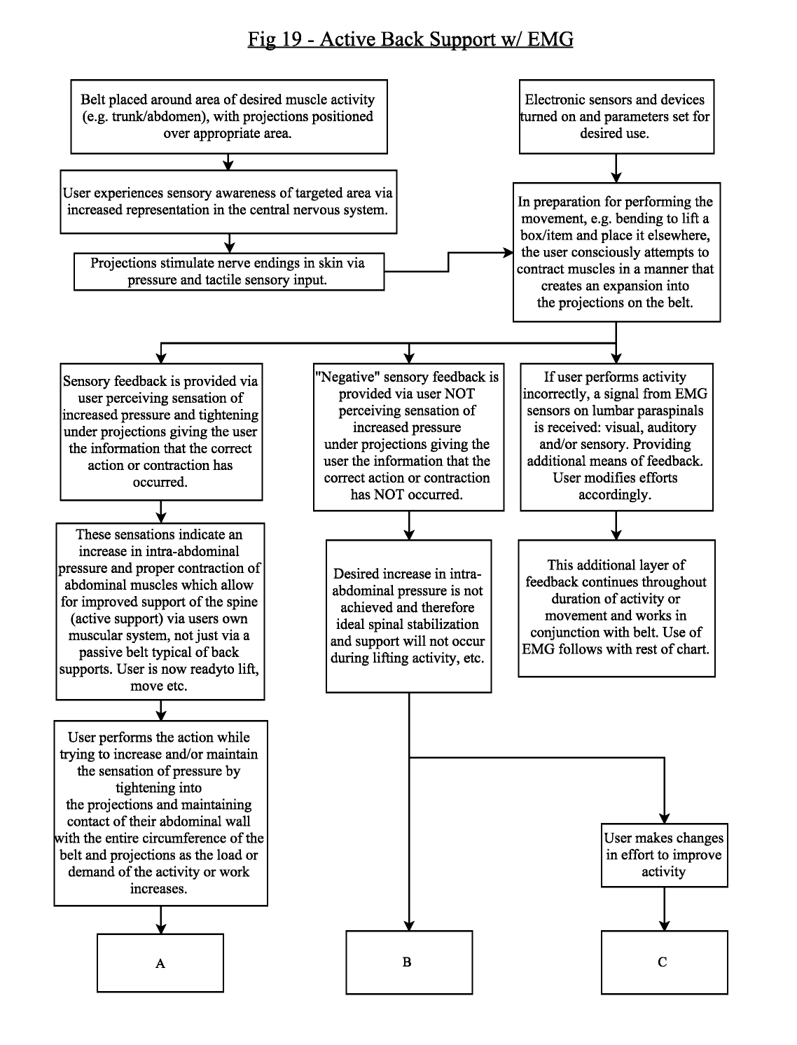

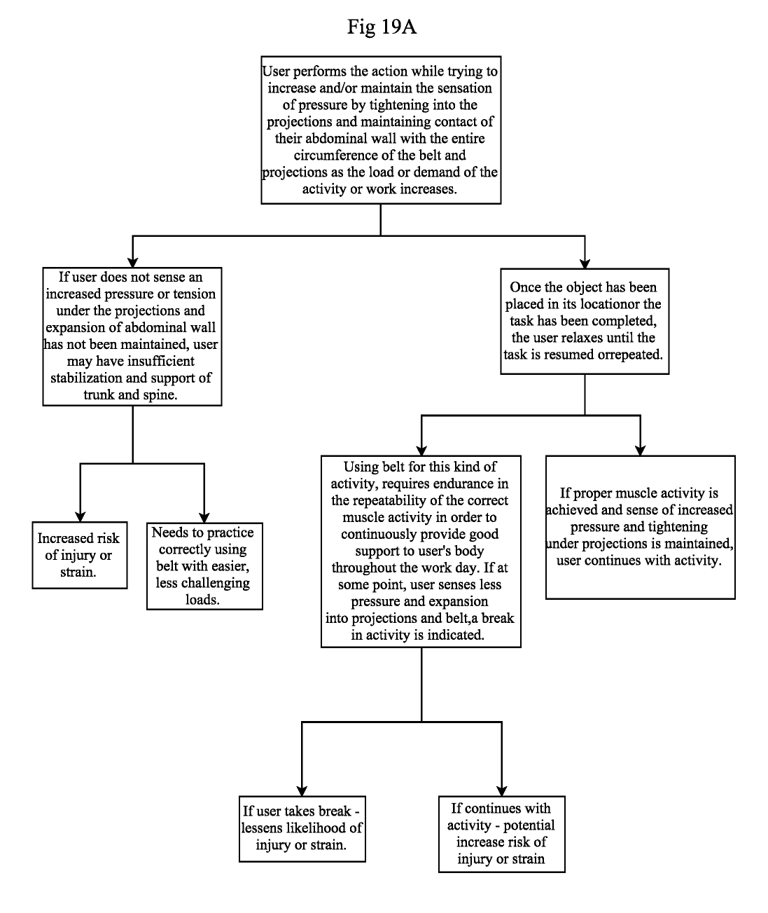

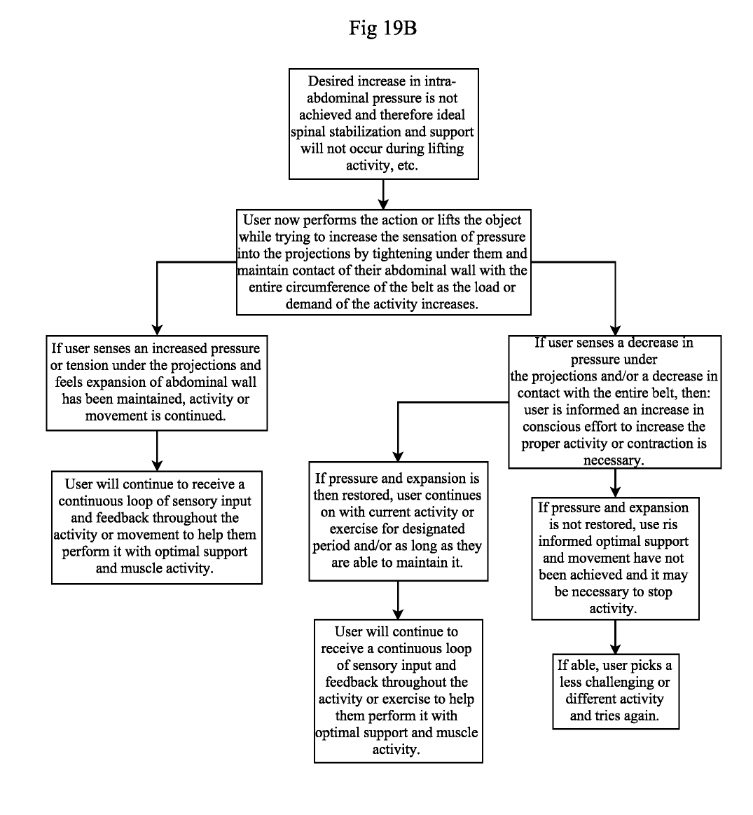

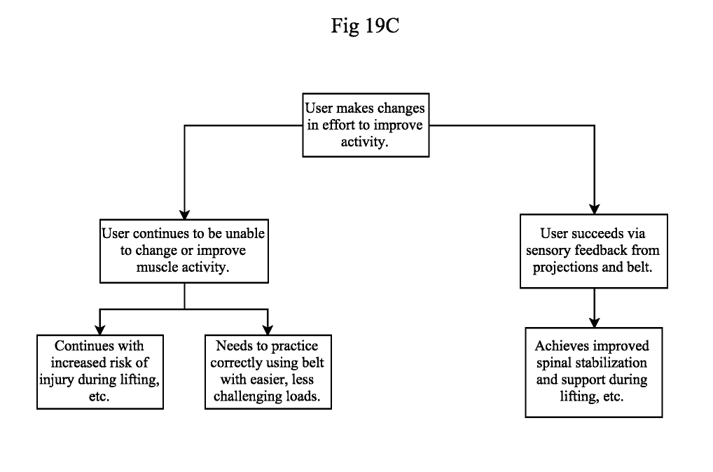

FIG. 19 is a block diagram of how to use the apparatus for back support in conjunction with electronic sensors;

FIG. 19A is a block diagram of how to use the apparatus for back support in conjunction with electronic sensors;

FIG. 19B is a block diagram of how to use the apparatus for back support in conjunction with electronic sensors;

FIG. 19C is a block diagram of how to use the apparatus for back support in conjunction with electronic sensors;

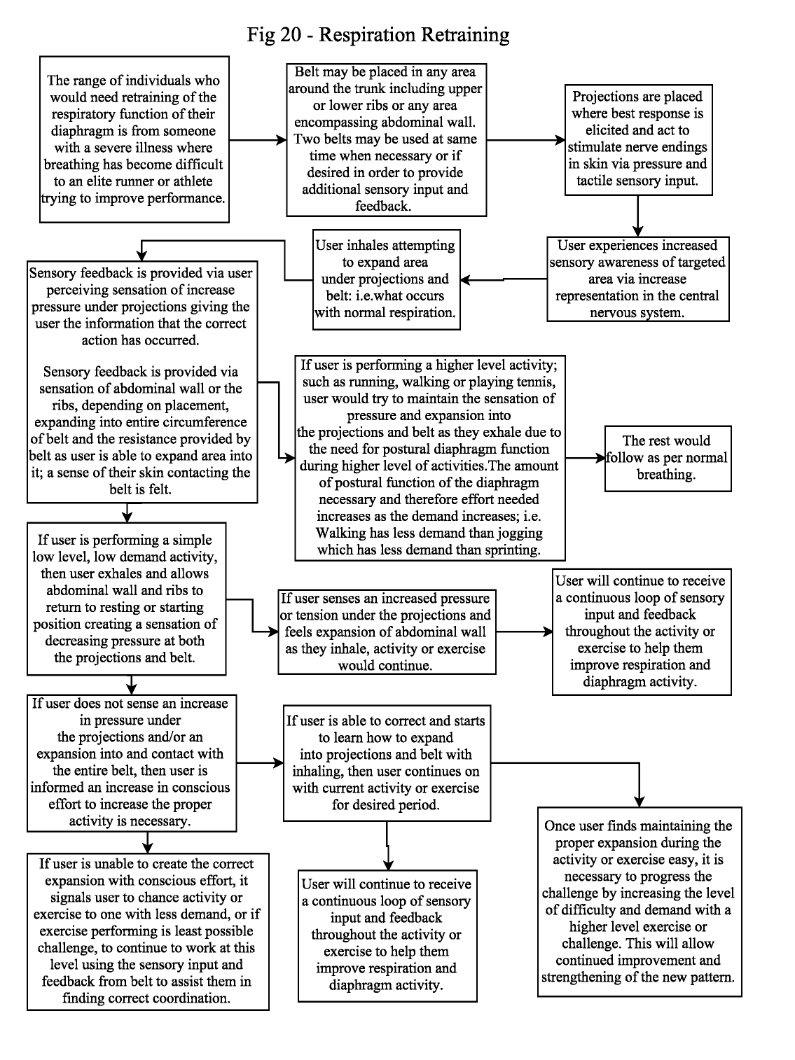

FIG. 20 is a block diagram of how to use the apparatus for respiration training;

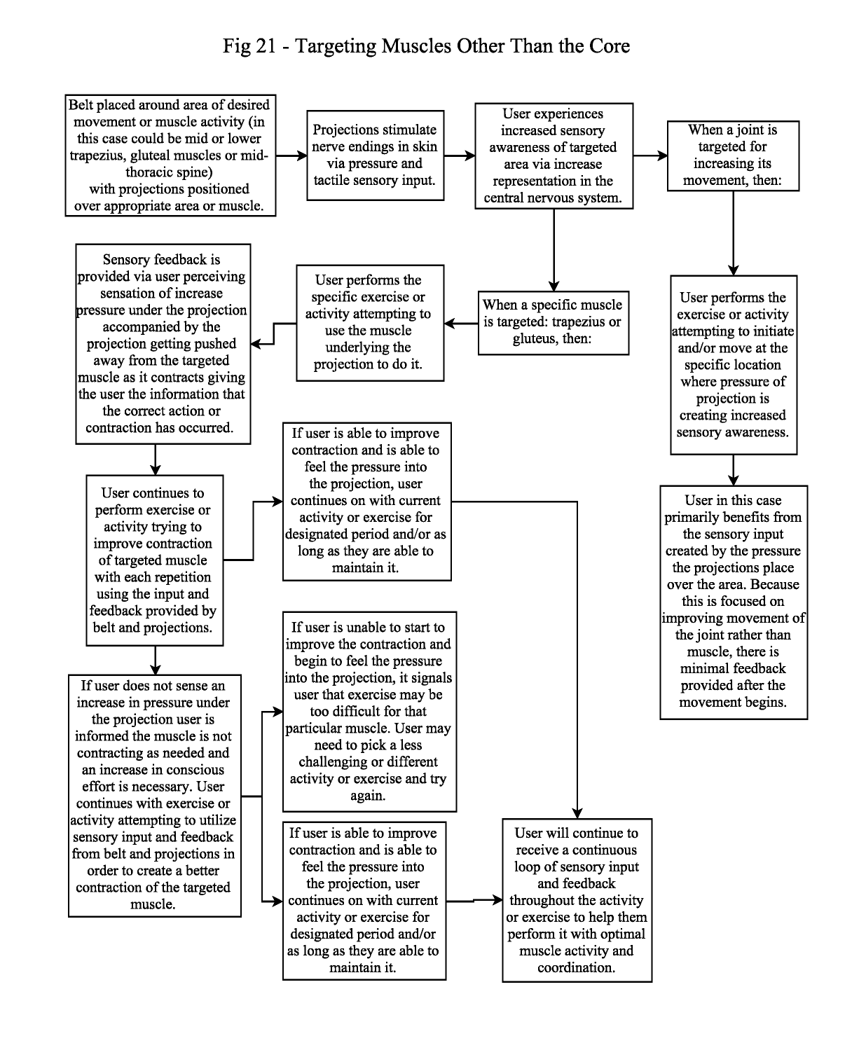

FIG. 21 is a block diagram of how to use the apparatus for targeting muscles and other areas other than core muscles; and,

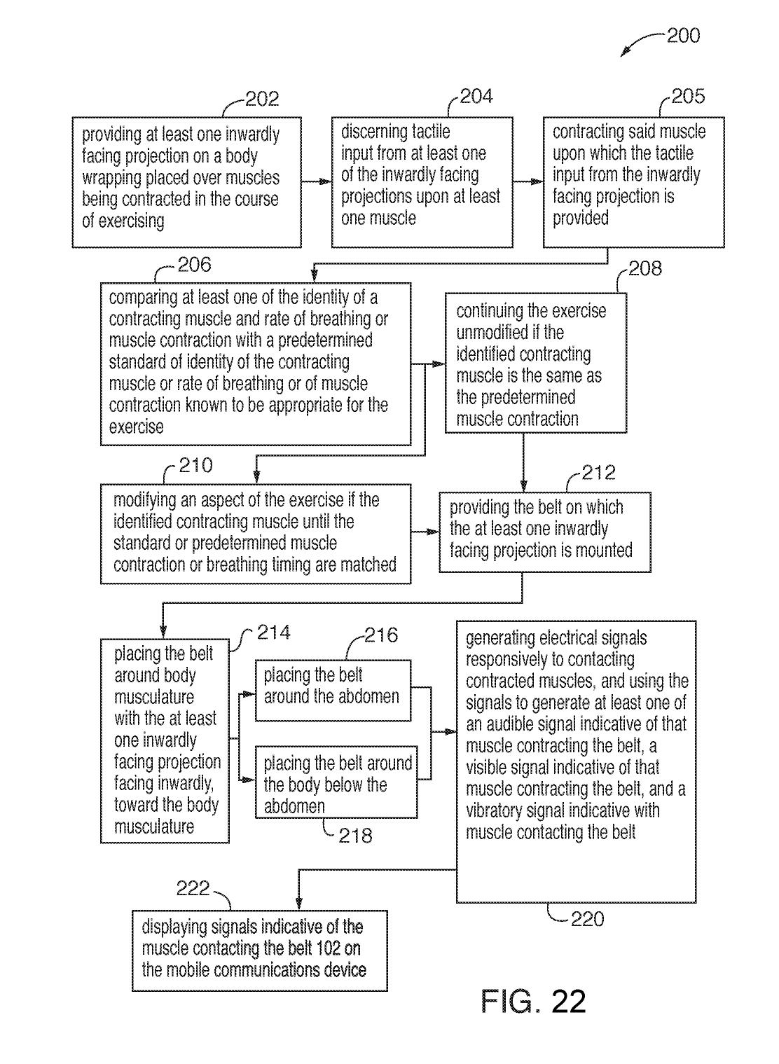

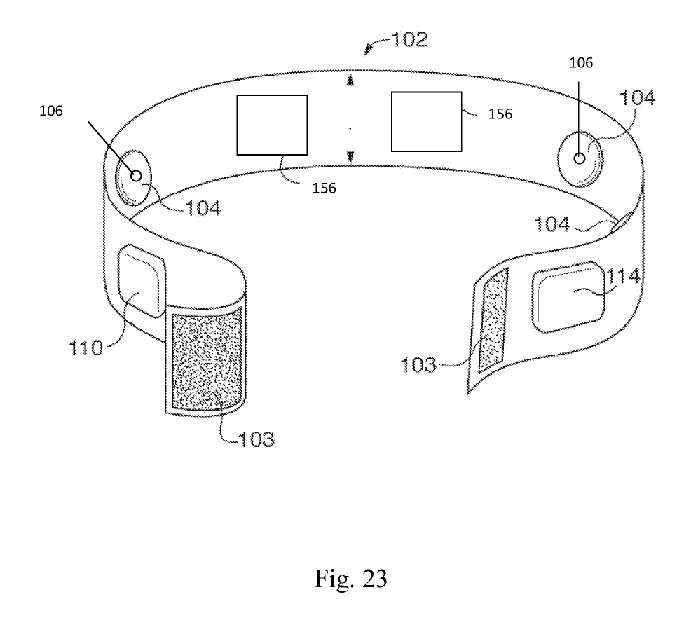

FIG. 22 is a block diagram of how to use the apparatus according to at least one aspect of the invention.

The above described apparatus and method(s) may be utilized for the user to improve the contraction and coordination of appropriate muscles and movement patterns necessary for efficient and optimal movement. Key areas which can be addressed include, the trunk or “core” muscles, proper diaphragm function, both respiratory and postural, as well as individual muscles such as the gluteus maximus and trapezius and the like. Improving these patterns in turn improves effective body movement during exercising, performing daily activities, and other demanding or functional movements. Notably, appropriate muscle contractions protect and stabilize the spine. This holds true not only for simple exercises, but for advanced exercises, walking, heavy lifting, running and the like.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

Moreover, the present invention and some of its advantages have been described in detail for some embodiments. It should be understood that although the process is described with reference to an apparatus and method to provide tactile sensations which allow the user to improve the quality of muscle activity and contraction during physical movement, in addition to enhancing respiratory functions, the process may be used in other contexts as well. It should also be understood that various changes, substitutions and alterations can be made herein without departing from the spirit and scope of the invention as defined by the appended claims. An embodiment of the invention may achieve multiple objectives, but not every embodiment falling within the scope of the attached claims will achieve every objective. Moreover, the scope of the present application is not intended to be limited to the particular embodiments of the process, machine, manufacture, composition of matter, means, methods and steps described in the specification. A person having ordinary skill in the art will readily appreciate from the disclosure of the present invention that processes, machines, manufacture, compositions of matter, means, methods, or steps, presently existing or later to be developed are equivalent to, and fall within the scope of, what is claimed. Accordingly, the appended claims are intended to include within their scope such processes, machines, manufacture, compositions of matter, means, methods, or steps.

I claim:

1. An apparatus for providing tactile input to a targeted area used in a physical activity prior to said area's contraction in order to enable the user to properly contract the targeted area during the physical activity, the apparatus comprising:
a body wrapping having elastic properties and having a body side and an exterior side;
at least one inwardly facing projection associated with the belt, wherein said projection is located on the body side of said body wrapping, and wherein when said body wrapping is worn by the user, the at least one inwardly facing projection is in contact with said targeted area and provides tactile input to said area prior to contraction; and,
at least one pressure sensor associated with said one inwardly facing projection for sensing and generating a signal.

2. The apparatus of claim 1, wherein the at least one pressure sensor senses and generates a signal when the targeted area for which tactile input is being provided by said projection contracts.

3. The apparatus of claim 1, wherein the at least one pressure sensor senses and generates a signal when the targeted area for which tactile input is being provided by said projection does not contract.

4. The apparatus of claim 1, wherein the projection is substantially semi-spherical having an x-axis defining a diameter and a y-axis defining a radius wherein the maximum diameter is 0.25 inches to 5 inches.

5. The apparatus of claim 1, wherein the projection is substantially semi-spherical having an x-axis defining a diameter and a y-axis defining a radius wherein the radius is 15% to 85% of the diameter.

6. The apparatus of claim 1, further comprising providing means for generating a vibration signal to said at least one projection.

7. The apparatus of claim 1, wherein the projection includes a nipple-like extension.

8. The apparatus of claim 1, further comprising a pouch having a loop portion for slideably attaching said sleeve onto said body wrapping, wherein said pouch is used to hold said projection and attach said projection to said body wrapping, wherein when said projection is held in said pouch, said projection faces and contacts said targeted area.

9. The apparatus of claim 1, further comprising a secondary exterior pressure enhancing band attached to said body wrapping.

10. The apparatus of claim 1, wherein the projection further includes an area on its surface which has a higher coefficient of friction relative to the material the projection is made from.

11. An apparatus for providing tactile input to a targeted area used in a physical activity prior to said area's contraction in order to enable the user to properly contract the targeted area during the physical activity, the apparatus comprising:
a body wrapping having elastic properties and having a body side and an exterior side; and,
at least one inwardly facing projection associated with the belt, wherein said projection is located on the body side of said body wrapping, and wherein when said body wrapping is worn by the user, the at least one inwardly facing projection is in contact with said targeted area and provides tactile input to said area prior to contraction.

12. The apparatus of claim 1, further comprising means for sensing and generating a signal when an area has contracted, wherein said area is not the targeted area for which tactile input is being provided by said projection.

13. The apparatus of claim 1, further comprising providing means for sensing and generating a signal when said targeted area has contracted.

14. The apparatus of claim 1, wherein the projection is substantially semi-spherical having an x-axis defining a diameter and a y-axis defining a radius wherein the maximum diameter is 0.25 inches to 5 inches.

15. The apparatus of claim 1, wherein the projection is substantially semi-spherical having an x-axis defining a diameter and a y-axis defining a radius wherein the radius is 15% to 85% of the diameter.

16. The apparatus of claim 1, further comprising providing means for generating a vibration signal to said at least one projection.

17. The apparatus of claim 1, wherein the projection includes a nipple-like extension.

18. The apparatus of claim 1, further comprising a pouch having a loop portion for slideably attaching said sleeve onto said body wrapping, wherein said pouch is used to hold said projection and attach said projection to said body wrapping, wherein when said projection is held in said pouch, said projection faces and contacts said targeted area.

19. The apparatus of claim 1, further comprising a secondary exterior pressure enhancing band attached to said body wrapping.

20. The apparatus of claim 1, wherein the projection further includes an area on its surface which has a higher coefficient of friction relative to the material the projection is made from.
